(19) United States
(12) Patent Application Publication
(10) Pub. No.: US 2007/0231838 A1
(43) Pub. Date: Oct. 4, 2007

(54) Method for the assay of rock kinase activity in cells

(76) Inventors: Andrew J. Garton, Farmingdale, NY (US); Linda Castaldo, Farmingdale, NY (US)

Correspondence Address: OSI PHARMACEUTICALS, INC., 41 PINELAWN ROAD, MELVILLE, NY, 11747, US

(21) Appl. No.: 11/732,168

(22) Filed: Apr. 3, 2007

Related U.S. Application Data
(60) Provisional application No. 60/788,926, filed on Apr. 3, 2006.

(51) Int. Cl. G01N 33/574; C12Q 1/48

(52) U.S. Cl. 435/7.23; 435/15

(57) ABSTRACT

The present invention provides a method for determining the intracellular activity of ROCK kinase comprising, providing a sample of cells to be tested for ROCK kinase activity, determining the level of phosphorylation of MYPT1 in the sample, and determining the intracellular activity of ROCK kinase in the sample of cells, wherein the level of MYPT1 phosphorylation directly correlates with the level of intracellular ROCK kinase activity. The invention further provides a method for identifying an agent that inhibits the intracellular activity of ROCK kinase comprising, providing a sample of cells having ROCK kinase activity, determining the degree of reduction of phosphorylation of MYPT1 in the sample by contacting the sample of cells with a test agent and comparing the MYPT1 phosphorylation level with the phosphorylation level of MYPT1 in an identical control sample of cells that was not contacted with the test agent, determining the degree of inhibition of intracellular activity of ROCK kinase in the sample of cells contacted with the agent, wherein the level of MYPT1 phosphorylation directly correlates with the level of intracellular ROCK kinase activity, and thus determining whether the test agent is an agent that inhibits the intracellular activity of ROCK kinase. The test agent may for example be a compound not known to have ROCK kinase inhibitory activity, or a compound identified by an in vitro ROCK kinase assay as having inhibitory activity.

### CROSS-REFERENCE TO RELATED APPLICATIONS

This application claims the benefit of U.S. Provisional Application No. 60/788,926 filed Apr. 3, 2006, which is herein incorporated by reference in its entirety.

### BACKGROUND OF THE INVENTION

Phosphoryl transferases are a large family of enzymes that transfer phosphorous-containing groups from one substrate to another. Kinases are a class of enzymes that function in the catalysis of phosphoryl transfer. The phosphorylation is usually a transfer reaction of a phosphate group from ATP to the protein substrate. Almost all kinases contain a similar 250-300 amino acid catalytic domain. Protein kinases, with at least 400 identified, constitute the largest subfamily of structurally related phosphoryl transferases and are responsible for the control of a wide variety of signal transduction processes within the cell. The protein kinases may be categorized into families by the substrates they phosphorylate (e.g., protein-serine/threonine, protein-tyrosine etc.). Protein kinase sequence motifs have been identified that generally correspond to each of these kinase families. Lipid kinases (e.g. PI3K) constitute a separate group of kinases with structural similarity to protein kinases.

The “kinase domain” appears in a number of polypeptides which serve a variety of functions. Such polypeptides include, for example, transmembrane receptors, intracellular receptor associated polypeptides, cytoplasmic located polypeptides, nuclear located polypeptides and subcellular located polypeptides. The activity of protein kinases can be regulated by a variety of mechanisms and any individual protein might be regulated by more than one mechanism. Such mechanisms include, for example, autophosphorylation, transphosphorylation by other kinases, protein-protein interactions, protein-lipid interactions, protein-polynucleotide interactions, ligand binding, and post-translational modification.

Phosphorylation of target proteins occurs in response to a variety of extracellular signals (hormones, neurotransmitters, growth and differentiation factors, etc.), cell cycle events, environmental or nutritional stresses, etc. Protein and lipid kinases regulate many different cell processes by adding phosphate groups to targets such as proteins or lipids. Such cell processes include, for example, proliferation, growth, differentiation, metabolism, cell cycle events, apoptosis, motility, transcription, translation and other signaling processes. Kinase catalyzed phosphorylation events act as molecular on/off switches to modulate or regulate the biological function of the target protein. Thus, protein and lipid kinases can function in signaling pathways to activate or inactivate, or modulate the activity (either directly or indirectly) of the targets. These targets may include, for example, metabolic enzymes, regulatory proteins, receptors, cytoskeletal proteins, ion channels or pumps, or transcription factors.

Protein kinases represent a large family of proteins which play a central role in the regulation of a wide variety of cellular processes, maintaining control over cellular function. Uncontrolled signaling due to defective control of protein phosphorylation has been implicated in a number of diseases and disease conditions, including, for example, inflammation, cancer, allergy/asthma, disease and conditions of the immune system, disease and conditions of the central nervous system (CNS), cardiovascular disease, dermatology, ocular diseases and angiogenesis.

Inappropriately high protein kinase activity has been implicated in many diseases resulting from abnormal cellular function. This might arise either directly or indirectly, by failure of the proper control mechanisms for the kinase, related to mutation, over-expression or inappropriate activation of the enzyme; or by over- or underproduction of cytokines or growth factors also participating in the transduction of signals upstream or downstream of the kinase. Alternatively, alterations in the activity of the phosphatases that normally serve to reverse the phosphorylation reaction may result in increased phosphdQrylation of the target proteins for a protein kinase. In all of these instances, selective inhibition of the action of the kinase might be expected to have a beneficial effect.

Initial interest in protein kinases as pharmacological targets was stimulated by findings that many viral oncogenes encode structurally modified cellular protein kinases with constitutive enzyme activity. One early example was the Rous sarcoma virus (RSV) or avian sarcoma virus (ASV), which caused highly malignant tumors of the same type or sarcomas within infected chickens. Subsequently, deregulated protein kinase activity, resulting from a variety of mechanisms, has been implicated in the pathophysiology of a number of important human disorders including, for example, cancer, CNS conditions, and immunologically related diseases. The development of selective protein kinase inhibitors that can block the disease pathologies and/or symptoms resulting from aberrant protein kinase activity has therefore become an important therapeutic target.

The Ser/Thr protein kinase family of enzymes comprises more than 400 members including 6 major subfamilies (AGC, CAMK, CMGC, GYC, TKL, STE). Many of these enzymes are considered targets for pharmaceutical intervention in various disease states.

ROCK1 and ROCK2 (rho-associated coiled-coil containing kinase-1 and -2, also known as Rokβ/p160ROCK and Rokα, respectively) are closely related members of the AGC subfamily of enzymes that are activated downstream of activated rho in response to a number of extracellular stimuli, including growth factors, integrin activation and cellular stress (Riento and Ridley, Nature Reviews Molecular Cell Biology, 4: 446-456 (2003)). The ROCK enzymes play key roles in multiple cellular processes including cell morphology, stress fiber formation and function, cell adhesion, cell migration and invasion, epithelial-mesenchymal transition (EMT), transformation, phagocytosis, apoptosis, neurite retraction, cytokinesis and mitosis and cellular differentiation (Riento and Ridley, Nature Reviews Molecular Cell Biology, 4: 446-456 (2003)). As such, ROCK kinases represent potential targets for development of inhibitors to treat a variety of disorders, including cancer, hypertension, vasospasm, asthma, preterm labor, erectile dysfunction, glaucoma, vascular smooth muscle cell hyperproliferation, atherosclerosis, myocardial hypertrophy, endothelial dysfunction and neurological diseases (Wettschurek and Offermanns, J Molecular Medicine, 80: 629-638 (2002); Mueller et al., Nature Reviews Drug Discovery, 4: 387-398 (2005), Sahai and Marshall, Nature Reviews Cancer, 2: 133-142 (2002)).

ROCK enzymes have been implicated in multiple disease processes, including cancer, glaucoma, cardiovascular disease and neurodegenerative diseases. Inhibition of ROCK activity reduces cell migration and reduces metastasis of tumor cells in vivo suggesting a potential role for ROCK in promoting cancer progression. via metastasis (Somlyo et al., Biochem Biophys Res Commun, 269: 6562-659 (2000); Somlyo et al., FASEB J, 17: 223-234 (2003); Genda et al., Hepatology, 30: 1027-1036 (1999; Takamura et al., Hepatology, 33: 577-581 (2001); Nakajima et al., Eur J Pharmacology, 459: 113-120 (2003); Nakaijima et al., Cancer Chemother Pharmacol, 52: 319-324 (2003); Itoh et al., Nature Medicine, 5: 221-225 (1999)). Overexpression of ROCK has been associated with invasion and metastasis in clinical samples derived from bladder cancer patients (Kamai et al., Clinical Cancer Research, 9: 2632-2641 (2003)) and ROCK protein is overexpressed in pancreatic cancer (Pancreas, 24: 251-257 (2002) and testicular cancer (Clin Cancer Res 10, 4799-4805 (2004)). Expression of constitutively active ROCK2 in colon cancer cells induced tumor dissemination into the surrounding stroma and increased tumor vascularity (Croft et al., Cancer Research 64, 8994-9001 (2004)). ROCK enzymes are involved in the transition of cells from an epithelial to mesenchymal phenotype (Bhowmick et al., Mol Biol Cell 12, 27-36 (2001)), a process thought to be important for progression of tumors towards a more malignant metastatic phenotype (Thiery, Nature Reviews Cancer, 2: 442-454 (2002)).

Many potential downstream substrates of ROCK have been suggested as a result of studies in a variety of cellular or in vivo systems. However, these substrates are also potentially recognized by several other protein Ser/Thr kinases such that the degree of phosphorylation observed is not necessarily an accurate reflection of the activity of the ROCK enzymes. For example, the regulatory light chain component of myosin (MLC) can be phosphorylated at Ser¹⁹ by ROCK (Amano et al., J. Biol. Chem. 271: 20246-20249 (1996); Wilkinson et al., Nature Cell Biol. 7: 255-261 (2005); Katoh et al., J. Cell Biol. 153: 569-583 (2001); Totsukawa et al., J. Cell Biol. 164: 427-439 (2004)), but under certain conditions is also a substrate for additional kinases, including myosin light chain kinases (MLCK) (Fazal et al., Mol. Cell. Biol. 25: 6259-6266 (2005); Katoh et al., J. Cell Biol. 153: 569-583 (2001); Totsukawa et al., J. Cell Biol. 164: 427-439 (2004)), citron kinase (CRIK) (Yamashiro et al., Mol. Biol. Cell 14: 1745-1756 (2003)), myotonic dystrophy-related and cdc42-activated kinases (MRCK) (Leung et al., Mol. Cell. Biol. 18: 130-140 (1998)), ZIPK (Niiro & Ikebe J. Biol. Chem. 276: 39567-29574 (2001); Murata-Hori et al., FEBS Lett. 451: 81-84 (1999); Tan et al. Mol. Cell. Biol. 21: 2767-2778 (2001); Wilkinson et al., Nature Cell Biol. 7: 255-261 (2005)). Similarly, LIMK can be phosphorylated at Thr⁵⁰⁵(LIMK1)/Thr⁵⁰⁸(LIMK2) by ROCK (Sumi et al., J. Biol. Chem. 276: 670-676 (2001); Maekawa et al., Science 285: 895-898 (1999); Ohashi et al., J. Biol. Chem. 275: 3577-3582 (2000)), but is also a direct substrate for p21-activated kinases (PAKs) (Misra et al., J Biol Chem. 280: 26278-26286 (2005); Edwards et al., Nature Cell Biol. 1: 253-259 (1999)). When phosphorylated at this site LIMK is activated to phosphorylate the downstream substrate Cofilin at Ser³ (Yang et al., Nature 393: 809-812 (1998); Soosairajah et al., EMBO J. 24: 473-86 (2005)).

The myosin binding subunit of protein phosphatase 1, MYPT1 (also known as MBS, MLCP) can be phosphorylated by ROCK at two major sites (Thr⁶⁹⁶ within the sequence RRSTQGV and T⁸⁵³ within the sequence RRSTGVS) (Velasco et al., FEBS Lett. 527: 101-104 (2002); Feng et al., J. Biol. Chem. 274: 37385-37390 (1999); Kimura et al., Science 273: 245-248 (1996); Kawano et al. J. Cell Biol. 147: 1023-1037 (1999); Wilkinson et al., Nature Cell Biol. 7: 255-261 (2005); Nagumo, H. et al. (2000) Am. J. Physiol. Cell Physiol. 278:C57-C65; Kitazawa, T. et al. (2003) J. Physiol. 546:879-889; Wilson, D. et al. (2005) Biochem J. 389:763-774; Kawano, Y. et al. (1999) J. Cell Biol. 147(5):1023-1037; Wardle, R. L. et al. (2006) J. Physiol. published online January 26, DOI:10.1113/jphysiol.2005.104083), but one or both of these sites are also recognized by several other enzymes, including myotonic dystrophy-related and cdc42-activated kinases (MRCK) (Tan et al., J. Biol. Chem. 276: 21209 (2001); Wilkinson et al., Nature Cell Biol. 7: 255-261 (2005)), ZIPK (MacDonald et al., Proc. Natl. Acad Sci. 98: 2419-2424 (2001), DMPK (Muranyi et al., FEBS Lett. 493: 80-84 (2001); Wansink et al., Mol. Cell. Biol. 23: 5489-5501 (2003)), integrin-linked kinase (ILK) (Kiss et al., Biochem. J. 365: 79-87 (2002); Muranyi et al., Biochem. J. 366: 211-216 (2002)) and Raf-1 (Broustas, C. G. et al., J. Biol. Chem. 277: 3053 (2002). Additional MYPT1-related proteins have also been identified, including MYPT2, MYPT3, MBS85, TIMAP and these proteins are thought to have related functions to MYPT1 in targeting protein phosphatase 1 catalytic subunits to myosin (Fujioka et al., Genomics 46: 59-68 (1998); Ito et al., Mol. Cell. Biochem. 259: 197-209 (2004); Banner et al., J. Biol. Chem. 278: 42190-42199 (2003); Cao et al., Am. J. Physiol. Cell Physiol. 283: C327-C337 (2002); Skinner & Saltiel Biochem. J. 356: 257-267 (2001)). Furthermore, MYPT2 and MBS85 are thought to be phosphorylated at one or more residues analogous to T696 and T853 on MYPT1 (Mulder et al., Mol. Biol. Cell 15: 5516-5527 (2004); Okamoto et al., Cell Signal. (2006)). These observations suggest that in certain cell types similar regulatory mechanisms may be involved in controlling phosphorylation and function of these proteins, and by analogy with MYPT1 may also be ROCK-dependent under certain circumstances.

Evaluation of the ability of novel compounds to inhibit their target proteins within the context of an intact cell is an important component in determining the utility of such compounds for treatment of disorders involving intracellular target proteins such as the Ser/Thr kinases ROCK1 and ROCK2. Ideally the method used to establish cell-based activity should be specific for the target protein of interest, and should be quantitative to allow identification of preferred compounds. However, despite advances in the development of compounds that can inhibit ROCK kinases, and the availability of multiple methods for assay of protein kinases (e.g. see International Patent Publication Nos. WO 95/14930; WO 03/087400; and WO 96/40276; and U.S. Pat. Nos. 5,763,198; 5,766,863; 5,580,742; 5,759,787; 5,686,310; 5,580,747; and 6,942,987), there remains a critical need for improved methods for evaluation of the activity of such compounds in cells. The invention described herein provides new specific assay methods that can rapidly and quantitatively determine the level of ROCK kinase activity in cells, either in vivo or in tissue culture, a result which has not previously been reported in the medical literature. Unlike previously reported assays, the ROCK kinase assay of the instant invention can directly measure the intracellular level of ROCK kinase activity.

### SUMMARY OF THE INVENTION

The present invention provides a method for determining the intracellular activity of ROCK kinase comprising, providing a sample of cells to be tested for ROCK kinase activity, determining the level of phosphorylation of MYPT1 in the sample, and determining the intracellular activity of ROCK kinase in the sample of cells, wherein the level of MYPT1 phosphorylation directly correlates with the level of intracellular ROCK kinase activity.

The invention further provides a method for identifying an agent that inhibits the intracellular activity of ROCK kinase comprising, providing a sample of cells having ROCK kinase activity, determining the degree of reduction of phosphorylation of MYPT1 in the sample by contacting the sample of cells with a test agent and comparing the MYPT1 phosphorylation level with the phosphorylation level of MYPT1 in an identical control sample of cells that was not contacted with the test agent, determining the degree of inhibition of intracellular activity of ROCK kinase in the sample of cells contacted with the agent, wherein the level of MYPT1 phosphorylation directly correlates with the level of intracellular ROCK kinase activity, and thus determining whether the test agent is an agent that inhibits the intracellular activity of ROCK kinase. The test agent may for example be a compound not known to have ROCK kinase inhibitory activity, or a compound identified by an in vitro ROCK kinase assay as having inhibitory activity.

### BRIEF DESCRIPTION OF THE FIGURES

FIG. 1: Expression of ROCK1 and ROCK2 in cancer cell lines: The indicated cell lines were cultured in their appropriate medium and lysed in RIPA buffer prior to analysis by SDS-PAGE and immunoblotting to evaluate the relative expression level of ROCK1 and ROCK2 proteins.

FIG. 2: Evaluation of ROCK1/ROCK2-dependence of cvtoskeletal signaling events in Panc1 cells using siRNA: Panc1 cells were transfected using with the indicated siRNA molecules (A) individually at either 10 nM or 100 nM, or (B) in combinations as indicated to achieve final total siRNA concentrations of 20-40 nM. Controls were either untreated, transfected with reagent only (no siRNA), or transfected with control siRNA with low or medium GC content. After 48 h incubation in standard growth medium (DMEM containing 1% L-glutamine, 10% FCS), lysates were prepared in SDS-PAGE sample buffer and aliquots were analyzed by immunoblotting with antibodies specific for ROCK1, ROCK2, GAPDH, pMYPT1(T696), pMYPT1(T853), pMLC(S19), pCofilin(S3). The signal intensity observed by immunoblotting was quantitated for each of the phospho-specific readouts and plotted relative to the signal intensity in control untreated cells (C): pMYPT1 (T853, filled bars; T696, open bars); (D): pMLC (S19, filled bars); pCofilin (S3, open bars).

FIG. 3: Extraction of cytoskeletal substrates of ROCK1/ROCK2 from Pancd cell extracts: (A) Panc-1 cells were incubated in the presence and absence of 10 μM ROCK inhibitor Y27632 for 1 h followed by lysis in the indicated buffers. Equal fractions of cell lysate were analyzed by immunoblotting for pMYPT1 (T696 or T853), total MYPT1, pMLC (S19) and total MLC. (B) 10 μL aliquots of Panc-1 cell lysate prepared in PROTEOEXTRACT® buffer (or buffer alone for control wells) were diluted in buffer A as indicated prior to addition to MYPT1 antibody-coated ELISA assay plate wells. Quantitation of the extent of MYPT1 (T853) phosphorylation was performed in triplicate assay wells as described in the methods section. Results plotted are mean signal intensities, error bars represent Standard Deviations.

FIG. 4: Time course of reduction of MYPT1 phosphorylation bv ROCK inhibitors in Pancl cells: Panc-1 cells were incubated in the presence and absence of 10 μM ROCK inhibitor Y27632 for the indicated time points followed by lysis in SDS-PAGE sample buffer. Equal fractions of cell lysate were then analyzed by immunoblotting for pMYPT1 (T696 or T853) and total MYPT1 protein.

FIG. 5: Effect of ROCK inhibitors on MYPT1 (T853) phosphorylation in Panc-1 cells: (A) Panc-1 cells were incubated in the presence and absence of the indicated concentrations of ROCK inhibitors H1152, Y27632 or HA1077 for 1 h, followed by lysis in SDS-PAGE sample buffer. Equal fractions of cell lysate were then analyzed by immunoblotting for pMYPT1 (T696 or T853) and total MYPT1 protein. (B) Panc1 cells were grown in 96-well culture plates, and incubated for 1 h with the indicated concentrations of compound ROCK inhibitors H1152, Y27632 or HA1077. Lysates were then prepared in PROTEOEXTRACT® buffer, and analyzed in the pMYPT1 (T853) ELISA assay as described in the Methods section.

FIG. 6: MYPT1 (T853) phosiphorylation assay comparison across a panel of cell lines: The indicated cell lines were incubated in normal growth medium (or in 90% human plasma—open bar) in the presence of ROCK inhibitor H1152 for 1 h prior to lysis and evaluation of pMYPT1 (T853) content in the quantitative ELISA. Data plotted are IC₅₀ values obtained from sigmoidal dose-response inhibition curves.

FIG. 7: MYPT1 (T696) phosphorylation assay in tumor samples: The indicated cell lines were grown as tumor xenografts in immuno-compromised mice to a size range 200-400 mm³, at which point the tumor tissue was harvested and rapidly frozen until required for analysis. Tumors were then lysed in PROTEOEXTRACT®, and samples were analyzed by SDS-PAGE and immunoblotting for (A) pMYPT1 (T696), total MYPT1 and pMLC (S19); lysates from Panc-1 cells grown in culture were included as a control. The same samples were also diluted 10-fold in Triton lysis buffer, and pMYPT1 content was quantitated by transferring the indicated volumes of lysate to an ELISA plate pre-coated with MYPT1 antibody, followed by detection with a pMYPT1 (T696) antibody (B).

### DETAILED DESCRIPTION OF THE INVENTION

The term “cancer” in an animal refers to the presence of cells possessing characteristics typical of cancer-causing cells, such as uncontrolled proliferation, immortality, metastatic potential, rapid growth and proliferations rate, and certain characteristic morphological features. Often, cancer cells will be in the form of a tumor, but such cells may exist alone within an animal, or may circulate in the blood stream as independent cells, such as leukemic cells.

The term “ROCK kinase” as used herein, for example in referring to determination of the intracellular activity of “ROCK kinase”, is used to mean ROCK1 or ROCK2, or a combination of both of these kinases. The NCBI GeneID number, a unique identifier of a gene from the NCBI Entrez Gene database record (National Center for Biotechnology Information (NCBI), U.S. National Library of Medicine, 8600 Rockville Pike, Building 38A, Bethesda, Md. 20894; Internet address http://www.ncbi.nlm.nih.gov/), is 6093 for human ROCK1 and 9475 for human ROCK2. By ROCK1 or ROCK2 is meant any protein product expressed by these genes, or other homologous ROCK kinases from other mammalian species, that have kinase activity, including variants thereof, such as splice variants, co- and post-translationally modified proteins, polymorphic variants etc. The NCBI RefSeq (Reference Sequence), an example of a sequence expressed by the gene, is NP_{—}005397 for human ROCK1 and NP_{—}004841 for human ROCK2.

The term “antibody reagent” as used herein refers to an antibody preparation that can be used for the specific detection of an antigen. It can comprise individual polyclonal or monoclonal antibodies, immunoreactive fragments of these antibodies, or a cocktail of such antibodies or antibody fragments. As described in further detail herein, for quantitative detection of antigen these antibodies or antibody fragments are labeled directly with a reporter or indirectly with a member of a specific binding pair using conventional techniques.

“Cell growth”, as used herein, for example in the context of “tumor cell growth”, unless otherwise indicated, is used as commonly used in oncology, where the term is principally associated with growth in cell numbers, which occurs by means of cell reproduction (i.e. proliferation) when the rate the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or over-expression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The phrase “a method of treating” or its equivalent, when applied to, for example, cancer refers to a procedure or course of action.that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

Using siRNA oligonucleotides directed towards the ROCK proteins, data presented in the Examples herein below demonstrate that the degree of MYPT1 phosphorylation at T853 or T696 in cells correlates with the activity of ROCK enzymes within cells, despite the fact that one or both of these phosphorylation sites are also recognized by several other protein kinases. Taking advantage of this surprising discovery has enabled the development of methods for quantitation of phosphorylated MYPT1, and thus the intracellular steady-state level of ROCK kinase activity of cells. It has been demonstrated that these methods can be used in a high-throughput manner to compare ROCK kinase inhibitor compound potency in a variety of cell types. It has also been demonstrated that the method is amenable for use in the evaluation of MYPT1 phosphorylation levels, and thus intracellular ROCK kinase activity, in tissue samples (e.g. tumor tissue), and thus is useful for the study of ROCK kinase inhibitors in both animal studies and clinical evaluation. Thus, the invention described herein provides new specific assay methods that can rapidly and quantitatively determine the level of ROCK kinase activity in cells, either in vivo or in tissue culture. The ROCK kinase assay methods of the instant invention directly measure the intracellular steady-state level of ROCK kinase activity (i.e. the intracellular steady-state level of ROCK kinase activity at the MYPT1 phosphorylation sites resulting from a balance between the ROCK kinases and protein phosphatases acting on these sites).

Accordingly, the present invention provides a method for determining the intracellular activity of ROCK kinase comprising, providing a sample of cells to be tested for ROCK kinase activity, determining the level of phosphorylation of MYPT1 in the sample of cells, and determining the intracellular activity of ROCK kinase in the sample of cells, wherein the level of MYPT1 phosphorylation directly correlates with the level of intracellular ROCK kinase activity (i.e. high levels of MYPT1 phosphorylation directly correlate with high intracellular ROCK kinase activity; and low levels of MYPT1 phosphorylation directly correlate with low intracellular ROCK kinase activity). Determining the intracellular activity of ROCK kinase in a test sample of cells from the level of MYPT1 phosphorylation can for example be done by reference to one or more other samples with different levels of MYPT1 phosphorylation in order to determine the relative level of ROCK kinase activity in the sample, e.g. by use of a calibration curve. In one embodiment of this method the level of phosphorylation of MYPT1 in the sample at the phosphorylation site threonine 853 is determined. In another embodiment of this method the level of phosphorylation of MYPT1 in the sample at the phosphorylation site threonine 696 is determined. In another embodiment of this method the level of phosphorylation of MYPT1 in the sample at the sum of both of the phosphorylation sites threonine 853 and threonine 696 is determined. In the latter embodiment the level of phosphorylation of MYPT1 in the sample may be determined by employing a mixture of reagents (e.g. antibodies) that each have specificity for only one phosphorylation site, or a reagent (e.g. an antibody) that is specific to a consensus sequence and thus interacts with either of the two MYPT1 phosphorylation sites.

In the context of this invention, the phosphorylation sites at “threonine 853” and “threonine 696” of MYPT1 refer to the two major phophorylation sites in MYPT1 (i.e. Thr⁸⁵³ within the sequence RRSTGVS and Thr⁶⁹⁶ within the sequence RRSTQGV in human MYPT1; or the equivalent phosphorylation sites in other animal MYPT1 proteins) (Velasco et al., FEBS Lett. 527: 101-104 (2002); Feng et al., J. Biol. Chem. 274: 37385-37390 (1999); Kimura et al., Science 273: 245-248 (1996); Kawano et al. J. Cell Biol. 147: 1023-1037 (1999); Wilkinson et al., Nature Cell Biol. 7: 255-261 (2005)). It should be noted that due to amino acid numbering differences between species or the presence of different splice forms of the gene product the amino acid residue numbers used for these phosphorylation sites may vary in the scientific literature, and thus the sequence context should be used as the primary determinant of the correct phosphorylation site, rather than the residue number per se.

In one embodiment of the method of the invention, the level of phosphorylation of MYPT1 is determined using a sandwich ELISA assay in which a first antibody reagent is specific to MYPT1 protein and a second antibody reagent is specific to one or more ROCK kinase phosphorylation sites on MYPT1. In one example of this embodiment, the second antibody reagent is specific to the ROCK kinase phosphorylation site on MYPT1 at threonine 853. In another example of this embodiment, the second antibody reagent is specific to the ROCK kinase phosphorylation site on MYPT1 at threonine 696. In yet another example of this embodiment, the second antibody reagent is specific to the ROCK kinase phosphorylation sites on MYPT1 at threonine 853 and threonine 696. In a preferred embodiment of these methods the first antibody reagent is adsorbed onto a surface (e.g. a plate or dish, e.g. a 96-well plate), a cell extract is prepared from the sample of cells to be tested such that the MYPT1 protein is solubilized (e.g. using a protein solubilizing agent such as SDS or PROTEOEXTRACT®), the cell extract is treated if necessary to ensure that any agents used to solubilize MYPT1 will not affect antibody binding to MYPT1 (e.g. by dilution, addition of detergents such as Triton X-100), MYPT1 protein in the extract is adsorbed onto the surface by contacting with the first antibody, and the phosphorylation level of the adsorbed MYPT1 is quantitated by contacting with a labeled second antibody reagent that is specific to one or more ROCK kinase phosphorylation sites on MYPT1. Due to their speed and simplicity, such ELISA methods are particularly advantageous where a rapid assay of ROCK kinase activity is required, or where large numbers of sample have to be analyzed, e.g. in a high-throughput compound screen. ELISA methods are well known to those of skill in the art, e.g. see International Patent Publication No. WO 95/14930, or Using Antibodies, A Laboratory Manual, edited by Harlow, E. and Lane, D., 1999, Cold Spring Harbor Laboratory Press, (e.g. ISBN 0-87969-544-7).

In performing the methods of the instant invention, it should be noted that due to the fact that MYPT1 is largely associated with insoluble fractions in cell extracts, a protein solubilizing agent such as SDS is used to solubilize MYPT1 and make it amenable to immunoassay methods such as those described above (e.g. ELISA). Consequently, prior to contacting the solubilized MYPT1 with antibody reagents used to quantitate its level of phosphorylation, it may be necessary to treat cell extracts containing such a protein solubilizing agent in order to “neutralize” the effects of the solubilizing agent, which may cause denaturation of some antibody reagents. This is readily achieved by, for example, dilution and/or addition of other detergents (e.g. Triton X-100; Tween-20; deoxycholate), or by removing the solubilizing agent (e.g. by adsorption of the MYPT1 onto a membrane prior to immunoblotting analysis).

In alternate embodiments of the instant invention other standard immunoassay formats may be used in place of the sandwich ELISA assay format for the determination of the level of phosphorylated MYPT, e.g. an antigen competition assay with phosphorylated MYPT1 adsorbed onto a solid phase (e.g. a 96-well plate), with the amount of phosphorylated MYPT1 in the sample being quantitated by its competition with the solid phase bound MYPT1 for binding to a labeled phosphorylation-site-specific antibody in solution.

In another embodiment, a dot blot assay may be used for the determination of the level of phosphorylated MYPT. Accordingly, the latter embodiment provides a method for determining the intracellular activity of ROCK kinase comprising, providing a sample of cells to be tested for ROCK kinase activity, determining the level of phosphorylation of MYPT1 in the sample by solubilizing the MYPT1 protein in the cell sample, adsorbing the MYPT1 protein onto a membrane (e.g. a hydrophobic membrane, nitrocellulose, nylon), and contacting with a labeled antibody reagent that is specific to one or more ROCK kinase phosphorylation sites on MYPT1, and determining the intracellular activity of ROCK kinase in the sample of cells, wherein the level of MYPT1 phosphorylation directly correlates with the level of intracellular ROCK kinase activity.

In another embodiment of the method of the invention, the level of phosphorylation of MYPT1 is determined by electrophoretic separation of the proteins in the sample and immunoblot analysis using an antibody reagent specific to one or more ROCK kinase phosphorylation sites on MYPT1. In one example of this embodiment, the antibody reagent is specific to the ROCK kinase phosphorylation site on MYPT1 at threonine 853. In another example of this embodiment, the antibody reagent is specific to the ROCK kinase phosphorylation site on MYPT1 at threonine 696. In yet another example of this embodiment, the antibody reagent is specific to the ROCK kinase phosphorylation sites on MYPT1 at threonine 853 and threonine 696. In a preferred embodiment of the above methods, electrophoretic separation of the proteins in the sample is achieved by SDS-PAGE.

In another embodiment of the method of the invention, the level of phosphorylation of MYPT1 is determined using an immunostaining procedure with an antibody reagent that is specific to one or more ROCK kinase phosphorylation sites on MYPT1. In one example of this embodiment, the antibody reagent is specific to the ROCK kinase phosphorylation site on MYPT1 at threonine 853. In another example of this embodiment, the antibody reagent is specific to the ROCK kinase phosphorylation site on MYPT1 at threonine 696. In yet another example of this embodiment, the antibody reagent is specific to the ROCK kinase phosphorylation sites on MYPT1 at threonine 853 and threonine 696. In one embodiment the immunostaining procedure is immunofluorescent detection of phosphorylated MYPT1, using for example cultured cells in a flask or plate, or cell smears from tissue samples, biopsies or needle aspirates. In another embodiment the immunostaining procedure is immunohistochemical detection of phosphorylated MYPT1, using for example cell smears from tissue samples, biopsies or needle aspirates, or tissue sections that have been fixed to preserve the tissue structure, e.g. by freezing, or by paraformaldehyde fixation and paraffin embedding. Standard methods for cell or tissue fixation, binding of antibody reagents, and labeling or staining can be employed in these immunostaining procedures (e.g. see Using Antibodies, A Laboratory Manual, edited by Harlow, E. and Lane, D., 1999, Cold Spring Harbor Laboratory Press (e.g. ISBN 0-87969-544-7), particularly chapters 5 and 6 on staining cells and tissues).

The present invention further provides a method for determining the intracellular activity of ROCK kinase comprising (a) providing a sample of cells to be tested for ROCK kinase activity, (b) treating the sample with a reagent in order to solubilize the ROCK kinase substrate MYPT1, (c) determining the level of phosphorylation of MYPT1 in the treated sample, for example by using a sandwich ELISA assay in which a first antibody reagent is specific to MYPT1 protein and a second antibody reagent is specific to one or more ROCK kinase phosphorylation sites on MYPT1, or by electrophoretic separation of the proteins in the sample and immunoblot analysis using an antibody reagent specific to one or more ROCK kinase phosphorylation sites on MYPT1, and (d) determining the intracellular activity of ROCK kinase in the sample of cells, wherein the level of MYPT1 phosphorylation directly correlates with the level of intracellular ROCK kinase activity. In one embodiment the treatment of the sample with a reagent in order to solubilize the ROCK kinase substrate MYPT1 comprises treatment with a protein-denaturing detergent, e.g. SDS. In another embodiment the treatment of the sample with a reagent in order to solubilize the ROCK kinase substrate MYPT1 comprises treatment with a chaotropic agent, e.g. urea, guanidinium hydrochloride. In another embodiment the treatment of the sample with a reagent in order to solubilize the ROCK kinase substrate MYPT1 comprises treatment with the commercially available protein solubilizing reagent termed PROTEOEXTRACT® (Calbiochem, San Diego, Calif.).

In an embodiment of the methods of the invention the sample of cells is a sample of cells from cells grown in a tissue culture dish, plate or flask, e.g. a multi-well plate (e.g. 96-well). The cells may be grown for example in monolayer, suspension, or on beads. Examples of suitable cells include Panc-1, HCT116, PC3, DU-145, A375, Geo, TENN, WBA, A1165, or ES-2 cells. In another embodiment of the method of the invention the sample of cells is, or is obtained from, a tissue biopsy, e.g. a tumor biopsy. For example the sample of cells may be from tumors or tumor metastases associated with any of the cancers listed herein (e.g. see Paragraph 65), e.g. a pancreatic tumor, a breast tumor, a colon tumor, a NSCL tumor, etc. The sample of cells may also be from tissues associated with other diseases involving ROCK kinase, e.g. eye tissues, vascular tissues, neural tissue.

In an alternative embodiment of any of the methods of the instant invention, the sample of cells-to be tested for ROCK kinase activity can be engineered to express recombinant MYPT1 protein. The recombinant MYPT1 protein may be expressed with a protein tag or as a fusion protein (e.g. hexahistidine, glutathione S-transferase (GST), maltose binding protein (MBP)). In such embodiments, for the determination of the level of phosphorylated MYPT1, it is thus possible to use antibody reagents that are specific to the tag or non-MYPT1 part of such fusion proteins to substitute for antibody reagents that are specific for MYPT1. The recombinant MYPT1 protein can be human, or from another animal species. In another alternative embodiment of any of the methods of the instant invention, the sample of cells to be tested for ROCK kinase activity can be engineered to express one or more recombinant proteins with one or more copies of one or both of the two major MYPT1 phosphorylation sites that can be phosphorylated by ROCK kinase (as described above). The recombinant protein sequence other than the phosphorylation site sequences can be from MYPT1 or any other unrelated protein. Examples of such recombinant proteins would include fragments of MYPT1 with at least one of the two major MYPT1 phosphorylation sites that can be phosphorylated by ROCK kinase. Such recombinant proteins would also include proteins where all or most of the sequence except the phosphorylation site(s) is from a protein or proteins that is not MYPT1. In a preferred example of these embodiments the sample of cells are cells grown in tissue culture. Examples of cells that may be used include CHO, cos7, or 293, or any other cell suitable for the expression of a recombinant protein. For expression of the recombinant proteins described above the polynucleotides encoding the proteins are cloned into expression vectors. Such expression vectors are routinely constructed in the art of molecular biology and may for example involve the use of appropriate initiators, promoters, enhancers and other elements, such as for example polyadenylation signals which may be necessary, and which are positioned in the correct orientation,. in order to allow for protein expression. Suitable vectors would be apparent to any person skilled in the art. By way of example in this regard, Molecular Cloning: a Laboratory Manual, 2001, 3rd Edition, by Joseph Sambrook and Peter MacCallum (the former Maniatis Cloning manual) provides a good source.

Accordingly, the present invention provides a method for determining the intracellular activity of ROCK kinase comprising, providing a sample of cells to be tested for ROCK kinase activity, wherein the cells express a recombinant protein possessing one or more of the two major MYPT1 phosphorylation sites that can be phosphorylated by ROCK kinase in cells, determining the level of phosphorylation of the recombinant protein, and determining the intracellular activity of ROCK kinase in the sample of cells, wherein the level of phosphorylation of the recombinant protein directly correlates with intracellular ROCK kinase activity. Any of the methods described herein for determining the level of phosphorylation of MYPT1, or comparable or equivalent methods, may be used to determine the level of phosphorylation of the recombinant protein, e.g. a sandwich ELISA assay, using an antibody capture reagent with specificity for part of the sequence of the recombinant protein other than the site(s) that can be phosphorylated by ROCK kinase.

The methods of the instant invention can be used in a compound screen to identify new ROCK kinases, or to test the intracellular effects of known ROCK kinase inhibitors, either in tissue or organ culture, or in an in vivo setting. In an in vivo setting the methods of this invention can be used to determine tissue specific effects of ROCK kinase inhibitors in vivo, e.g. in pharmacodynamic and pharmacokinetic studies. For example, the ability of a ROCK kinase inhibitor to inhibit ROCK kinase in tumor cells can be determined in cells from a tumor biopsy. In one embodiment, the methods of the instant invention can be used in a high-throughput screen (HTS) of compounds to identify ROCK kinase inhibitors that have activity on whole cells. In another embodiment, the methods of the instant invention can be used to compare potencies of ROCK kinase inhibitor compounds by assaying two or more compounds over a range of concentrations under identical incubation conditions and comparing the relative potencies. In another embodiment, the methods of the instant invention can be used to determine the activity of individual ROCK kinases in cell samples, i.e. ROCK1 or ROCK2. This can readily be achieved by any of the methods of the instant invention by including the additional step of treating the cell sample with an siRNA specific to either ROCK1 or ROCK2 in order to ablate the activity of one of these enzymes, such that the activity of only one of the ROCK kinases will be determined by the assay method. This method can be used for example to determine the relative potency of different ROCK kinase inhibitor compounds on individual ROCK kinases by pre-treating different samples of cells with siRNAs specific to each of the two ROCK kinases prior to contacting the cell samples with each of the compounds.

It is contemplated that the methods of the invention as described herein will prove to be valuable methods to assist in the development of new drugs for treating various pathophysiological conditions such as chronic and acute inflammation, arthritis, autoimmune diseases, transplant rejection, graft versus host disease, bacterial, fungal, protozoan and viral infections, septicemia, AIDS, pain, psychotic and neurological disorders, including anxiety, depression, schizophrenia, dementia, mental retardation, memory loss, epilepsy, neurological disorders, neuromotor disorders, respiratory disorders, asthma, eating/body weight disorders including obesity, bulimia, diabetes, anorexia, nausea, hypertension, hypotension, vascular and cardiovascular disorders, ischemia, stroke, cancers, ulcers, urinary retention, sexual/reproductive disorders, circadian rhythm disorders, renal disorders, bone diseases including osteoporosis, benign prostatic hypertrophy, gastrointestinal disorders, nasal congestion, dermatological disorders such as psoriasis, allergies, Parkinson's disease, Alzheimer's disease, acute heart failure, angina disorders, delirium, dyskinesias such as Huntington's disease or Gille's de la Tourette's syndrome, among others.

In the practice of this invention, determination of the level of phosphorylation of MYPT1 may be achieved by immunoassay of the phosphorylated form(s) of MYPT1, using polyclonal or monoclonal antibodies. Immunoreactive fragments of these antibodies or a cocktail of antibodies can also be used to practice the invention. These antibodies can be labeled directly with a reporter or indirectly with a member of a specific binding pair using conventional techniques.

In the practice of this invention any of the commonly used immunoassay techniques may be used for isolation of MYPT1 protein, or quantitation of the phosphorylation of MYPT1 protein, including immunoprecipitation, immunoblotting (Western blotting), immunostaining of cultured cells or tissue sections (e.g. immunofluorescence, immunohistochemistry), and ELISA assays. In one preferred embodiment, an ELISA assay is used in which the MYPT1 protein is initially captured using an anti-MYPT1 antibody, and phosphorylation then assessed in a second step using a labeled anti-phospho-MYPT1 antobody. In another preferred embodiment, an anti-MYPT1 antibody is used for isolation of the MYPT1 protein, for example by immunoprecipitation, and quantitation of the phosphorylation of MYPT1 protein is assessed using a labeled anti-phospho-MYPT1 antibody. In another preferred embodiment, MYPT1 is separated from other proteins by gel electrophoresis, the separated proteins blotted onto a membrane (e.g. nitrocellulose), and a labeled anti-phospho-MYPT1 antibody is used to assess the level of phosphorylation of MYPT1.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic.

Non-immune binding pairs include systems wherein the two components share a natural affinity for each other but are not antibodies. Exemplary non-immune pairs are biotin-streptavidin, intrinsic factor-vitamin B₁₂, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin can be covalently coupled to antibodies by utilizing commercially available active derivatives. Some of these are biotin-N-hydroxy-succinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein can be coupled to protein amine groups using fluorescein isothiocyanate. Dinitrophenyl groups can be coupled to protein amine groups using 2,4-dinitrobenzene sulfate or 2,4-dinitrofluorobenzene. Other standard methods of conjugation can be employed to couple monoclonal antibodies to a member of a specific binding pair including dialdehyde, carbodiimide coupling, homofunctional crosslinking, and heterobifunctional crosslinking. Carboduimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional crosslinkers, including the bifunctional imidoesters and bifunctional N-hydroxysuccinimide esters, are commercially available and are employed for coupling amine groups on one substance to amine groups on another. Heterobifunctional crosslinkers are reagents which possess different functional groups. The most common commercially available beterobifunctional crosslinkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups.

The detectably-labeled antibody or detectably-labeled member of the specific binding pair is prepared by coupling to a reporter, which can be a radioactive isotope, enzyme, fluorogenic, chemiluminescent or electrochemical materials. Two commonly used radioactive isotopes are ¹²⁵I and ³H. Standard radioactive isotopic labeling procedures include the chloramine T, lactoperoxidase and Bolton-Hunter methods for ¹²⁵I and reductive methylation for ³H. The term “detectably-labeled” refers to a molecule labeled in such a way that it can be readily detected by the intrinsic properties of the label or by the binding to the label of another component, which can itself be readily detected.

Enzymes suitable for use in this invention include, but are not limited to, horseradish peroxidase, alkaline phosphatase, β-galactosidase, glucose oxidase, luciferases, including firefly and renilla, β-lactamase, urease, green fluorescent protein (GFP) and lysozyme. Enzyme labeling is facilitated by using dialdehyde, carboduimide coupling, homobifunctional crosslinkers and heterobifunctional crosslinkers as described above for coupling an antibody with a member of a specific binding pair.

The labeling method chosen depends on the functional groups available on the enzyme and the material to be labeled, and the tolerance of both to the conjugation conditions. The labeling method used in the present invention can be one of, but not limited to, any conventional methods currently employed including those described by Engvall and Pearlmann, Immunochemistry 8, 871 (1971), Avrameas and Ternynck, Immunochemistry 8, 1175 (1975), Ishikawa et al., J. Immunoassay 4(3):209-327 (1983) and Jablonski, Anal. Biochem. 148:199 (1985).

Labeling can be accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, according to the present invention, the antibody used to detect can be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above.

Moreover, the unlabeled detector antibody can be detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody can be labeled directly or indirectly using any of the approaches discussed above. For example, the anti-antibody can be coupled to biotin which is detected by reacting with the streptavidin-horseradish peroxidase system discussed above.

In one embodiment of this invention biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA can be used to effect chromogenic detection.

In one preferred immunoassay format for practicing this invention, a forward sandwich assay is used in which the capture reagent (e.g. anti-MYPT1 antibody) has been immobilized, using conventional techniques, on the surface of a support. Suitable supports used in assays include synthetic polymer supports, such as polypropylene, polystyrene, substituted polystyrene, e.g. aminated or carboxylated polystyrene, polyacrylamides, polyamides, polyvinylchloride, glass beads, agarose, or nitrocellulose.

In the practice of this invention,.determination of phosphorylation of MYPT1 may also be achieved by methods that directly or indirectly involve the binding of phosphorylated MYPT1 to a non-immune binding protein with which it normally interacts, e.g. the PDZ domain of interleukin-16 precursor proteins (Mulder J. et al., Mol. Biol. Cell 15: 5516 (2004), or the coiled-coil domain of p116Rip (Bannert, N. et al., J. Biol. Chem. 278: 42190 (2003), both of which have been reported to bind to MYPT1 in regions outside of the phosphorylated portions of the protein. The association of the binding protein is monitored in a similar fashion as antibody binding.

Further, in the practice of this invention, an alternative detection method for phosphorylated MYPT1, particularly when assay of immunoaffinity purified phosphorylated MYPT1 protein is contemplated, is an assay whereby phosphorylated MYPT1 is monitored by the incorporation of radiophosphorus (e.g.³²P) into the phosphorylated MYPT1, via intracellular phosphorylation in the presence of p³² phosphate. Alternatively, in another embodiment, phosphorylated MYPT1 can be monitored by immunoassay techniques as described above but using anti-phosphothreonine antibodies as a means of determining the level of phosphorylation of MYPT1, rather than antibodies specific to the different phosphorylation sites.

In an alternative embodiment of any of the methods of the instant invention, peptide or RNA aptamer reagents can be substituted for one or more of the antibody reagents used. Such aptamers can interact with proteins with:a specificity comparable to antibodies, and thus can substitute for antibody reagents in the determination of the level of phosphorylation of MYPT1. Methods for selecting an appropriate peptide or RNA aptamer that is specific to the protein of interest are well known in the art (e.g. see Buerger, C. et al. et al. (2003) J. Biol. Chem. 278:37610-37621; Chen, C-H. B. et al. (2003) Proc. Natl. Acad. Sci. 100:9226-9231; and Buerger, C. and Groner, B. (2003) J. Cancer Res.:Clin. Oncol. 129(12):669-675. Epub 2003 September 11; and the references cited therein).

The invention further provides a method for identifying an agent that inhibits the intracellular activity of ROCK kinase comprising, providing a sample of cells having ROCK kinase activity, determining the degree of reduction of phosphorylation of MYPT1 in the sample by contacting the sample of cells with a test agent and comparing the MYPT1 phosphorylation level with the phosphorylation level of MYPT1 in an identical control sample of cells that was not contacted with the test agent, determining the degree of inhibition of intracellular activity of ROCK kinase in the sample of cells contacted with the agent, wherein the level of MYPT1 phosphorylation directly correlates with the level of intracellular ROCK kinase activity, and thus determining whether the test agent is an agent that inhibits the intracellular activity of ROCK kinase. The test agent may for example be a compound not known to have ROCK kinase inhibitory activity, or a compound identified by an in vitro ROCK kinase assay as having inhibitory activity.

This invention further provides a method of screening a plurality of chemical compounds not known to inhibit ROCK kinase activity to identify a compound which inhibits ROCK kinase activity, which comprises contacting a sample of cells having ROCK kinase activity with the plurality of compounds not known to inhibit ROCK kinase activity, under conditions permitting inhibition by compounds known to inhibit ROCK kinase activity; determining the level of phosphorylation of MYPT1 in the sample; determining the intracellular activity of ROCK kinase in the sample of cells, wherein the level of MYPT1 phosphorylation directly correlates with the level of intracellular ROCK kinase activity; comparing the intracellular activity of ROCK kinase in the sample of cells with that in an identical control sample of cells that had not been treated with the plurality of compounds; and where inhibition of ROCK kinase activity by the plurality of compounds is observed, separately determine the inhibition of ROCK kinase activity of each compound included in the plurality of compounds, so as to thereby identify any individual compound included therein which inhibits ROCK kinase. Determination of the inhibition of ROCK kinase activity by each individual compound included in the plurality of compounds can be by any of the methods of the invention described herein, or any other methods known in the art for determination of the activity of ROCK kinase inhibitors.

This invention further provides a method for identification of an agent that causes a reduction in tumor growth in a subject, which method comprises contacting a subject with a test agent which inhibits ROCK kinase activity, identified by any of the methods described herein, and monitoring tumor growth, thereby determining whether the test agent is an inhibitor of tumor growth.

This invention further provides a method for identification of an agent that causes activation of tumor cell apoptosis in a subject, which method comprises contacting a subject with a test agent which inhibits ROCK kinase activity, identified by any of the methods described herein, and monitoring tumor cell apoptosis, thereby determining whether the test agent is activator of tumor cell apoptosis.

This invention further provides a method for identification of an agent that causes a reduction in tumor cell motility in a subject, which method comprises contacting a subject with a test agent which inhibits ROCK kinase activity, identified by any of the methods described herein, and monitoring tumor cell motility, thereby determining whether the test agent is an inhibitor of tumor cell motility.

This invention further provides a method for identification of an agent that causes a reduction in tumor cell invasion in a subject, which method comprises contacting a subject with a test agent which inhibits ROCK kinase activity, identified by any of the methods described herein, and monitoring tumor cell invasion, thereby determining whether the test agent is an inhibitor of tumor cell invasion.

This invention further provides a method for identification of an agent that causes a reduction in tumor cell metastasis in a subject, which method comprises contacting a subject with a test agent which inhibits ROCK kinase activity, identified by any of the methods described herein, and monitoring tumor cell metastasis, thereby determining whether the test agent is an inhibitor of tumor cell metastasis.

In the context of the above methods a “subject” can be a human or animal subject, e.g. an animal model for testing an agents's effectiveness, or a human subject in a clinical trial.

This invention further provides a modulator of tumor growth, apoptosis, cell motility, cell invasion, or metastasis identified by any of the methods described herein. This invention further provides the use of such a modulator in the manufacture of a medicament for the treatment of abnormal cell proliferation or cancer.

This invention further provides a method of preparing a composition comprising a chemical compound which inhibits ROCK kinase activity, which comprises identifying an chemical that inhibits the intracellular activity of ROCK kinase comprising, providing a sample of cells having ROCK kinase activity, determining the degree of reduction of phosphorylation of MYPT1 in the sample by contacting the sample of cells with a test chemical, determining the degree of inhibition of intracellular activity of ROCK kinase in the sample of cells, wherein the level of MYPT1 phosphorylation directly correlates with the level of intracellular ROCK kinase activity, identifying the test chemical as a chemical that inhibits the intracellular activity of ROCK kinase, and admixing the test chemical so identified, or a functional analog or homolog of said test chemical, with a carrier, thereby preparing said composition.

In another embodiment of the methods of this invention, the ROCK kinase assay methods described herein can be an intergral part; of a treatment regimen for patients with cancer that may benefit from treatment with a ROCK kinase inhibitor. Accordingly, the present invention provides a method for treating tumors or tumor metastases in a patient, comprising the steps of diagnosing a patient's likely responsiveness to a ROCK kinase inhibitor, by assessing the degree of inhibition of ROCK kinase by a ROCK kinase inhibitor in a sample of tumor cells biopsied from the patient by treatment of the biopsied tumor cell sample with the ROCK kinase inhibitor and using any one of the methods described herein to determine the degree of inhibition of ROCK kinase, wherein high ROCK kinase inhibition in tumor cells correlates with potential high therapeutic effectiveness of treatment of the patient by a ROCK kinase inhibitor, and administering to said patient a therapeutically effective amount of a ROCK kinase inhibitor if the patient is predicted to potentially respond to a ROCK kinase inhibitor.

It will be appreciated by one of skill in the medical arts that the exact manner of administering to said patient of a therapeutically effective amount of a ROCK kinase inhibitor following a diagnosis of a patient's likely responsiveness to a ROCK kinase inhibitor will be at the discretion of the attending physician. The mode of administration, including dosage, combination with other anti-cancer agents, timing and frequency of administration, and the like, may be affected by the diagnosis of a patient's likely responsiveness to a ROCK kinase inhibitor, as well as the patient's condition and history. Thus, patients diagnosed with tumors predicted to be relatively insensitive to a ROCK kinase inhibitor as a single agent may still benefit from treatment with a ROCK kinase inhibitor, either alone or in combination with other anti-cancer agents, or other agents that may alter a tumor's sensitivity to a ROCK kinase inhibitor. Similarly, patients diagnosed with tumors predicted to be relatively sensitive to a ROCK kinase inhibitor as a single agent may still benefit from treatment with a combination of a ROCK kinase inhibitor and other anti-cancer agents, or other agents that may alter a tumor's sensitivity to a ROCK kinase inhibitor.

The present invention further provides a method for treating tumors or tumor metastases in a patient as described above, comprising in addition to administering to the patient a therapeutically effective amount of a combination of a ROCK kinase inhibitor, administering one or more other cytotoxic, chemotherapeutic or anti-cancer agents, or compounds that enhance the effects of such agents.

In the context of this invention, other cytotoxic, chemotherapeutic or anti-cancer agents or treatments, or compounds that enhance the effects of such agents or treatments, include, for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6MP), 6-thiocguanine (6TG), cytarabine (Ara-C), 5-fluorouracil (5-FU), capecitabine (e.g. XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil, anti-hormonal agents such as steroid receptor antagonists, anti-estrogens such as tamoxifen, raloxifene, aromatase inhibiting 4(5)-imidazoles and other aromatase inhibitors, and antagonists for other non-permissive receptors, such as antagonists for RAR, RXR, TR, and VDR, angiogenesis inhibitors, including, for example, VEGFR inhibitors, tumor cell pro-apoptotic or apoptosis-stimulating agents, signal transduction inhibitors, such as for example HER2 receptor inhibitors, EGFR kinase inbibitors (e.g. erlotinib), inhibitors of other protein tyrosine-kinases (e.g. imitinib, PDGFR inhibitors), ras inhibitors; raf inhibitors; MEK inhibitors; mTOR inhibitors; cyclin dependent kinase inhibitors; protein kinase C inhibitors; and PDK-1 inhibitors, inhibitors of the enzyme farnesyl protein transferase, COX II (cyclooxygenase II ) inhibitors, treatment with radiation or a radiopharmaceutical, agents capable of enhancing antitumor immune responses.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells, or a histocultured tissue sample, or the responses observed in the appropriate animal models.

As used herein, the term “patient” preferably refers to a human in need of treatment with a ROCK kinase inhibitor for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with a ROCK kinase inhibitor.

In a preferred embodiment, the patient is a human in need of treatment for cancer, or a precancerous condition or lesion, wherein the cancer is preferably NSCL, breast, colon or pancreatic cancer. In addition, other cancers that may be treated by the methods described herein include examples of the following cancers that are potentially treatable by administration of a ROCK kinase inhibitor: lung cancer, bronchioloalveolar cell lung cancer, bone cancer, skin cancer, cancer of the head or neck, cutaneous or intraocular melanoma, uterine cancer, ovarian cancer, rectal cancer, cancer of the anal region, stomach cancer, gastric cancer, uterine cancer, carcinoma of the fallopian tubes, carcinoma of the endometrium, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the ureter, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, and cancer of the kidney or renal cell carcinoma, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

The ROCK kinase inhibitor will typically be administered to the patient in a dose regimen that provides for the most effective treatment of the cancer (from both efficacy and safety perspectives) for which the patient is being treated, as known in the art (e.g. Hirooka, Y. et al. (2005) Am. J. Cardiovasc. Drugs 5(1):31-39; Lai, A. et al. (2005) Cardiol. Rev. 13(6):285-292; Vicari, R. M. et al. (2005) J. Am. Coll. Cardiol. 46(10):1803-1811; Shibuya, M. et al. (2005) J. Neurol. Sci. 238(1-2):31-39; Kishi, T. et al. (2005) Circulation 111(21):2741-2747). In conducting the treatment method of the present invention, the ROCK kinase inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of ROCK kinase inhibitor being used (for example, small molecule, RNAi, ribozyme or antisense construct), and the medical judgement of the prescribing physician as based, e.g., on the results of published clinical studies.

The amount of ROCK kinase inhibitor administered and the timing of ROCK kinase inhibitor administration will depend on the type (species, gender, age, weight, etc.) and condition of the patient being treated, the severity of the disease or condition being treated, and on the route of administration. For example, small molecule ROCK kinase inhibitors can be administered to a patient in doses ranging from 0.001 to 100 mg/kg of body weight per day or per week in single or divided doses, or by continuous infusion (see for example Hirooka, Y. et al. (2005) Am. J. Cardiovasc. Drugs 5(1):31-39; Lai, A. et al. (2005) Cardiol. Rev. 13(6):285-292; Vicari, R. M. et al. (2005) J. Am. Coll. Cardiol. 46(10):1803-1811; Shibuya, M.et al. (2005) J. Neurol. Sci. 238(1-2):31-39; Kishi, T. et al. (2005) Circulation 111(21):2741-2747). Antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The ROCK kinase inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

Methods of preparing pharmaceutical compositions comprising an ROCK kinase inhibitor are known in the art (e.g. Hirooka, Y. et al. (2005) Am. J. Cardiovasc. Drugs 5(1):31-39; Lai, A. et al. (2005) Cardiol. Rev. 13(6):285-292; Vicari, R. M. et al. (2005) J. Am. Coll. Cardiol. 46(10):1803-1811; Shibuya, M. et al. (2005) J. Neurol. Sci. 238(1-2):31-39; Kishi, T. et al. (2005) Circulation 111 (21):2741-2747). In view of the teaching of the present invention, methods of preparing pharmaceutical compositions comprising ROCK kinase inhibitors will be apparent from the above-cited publications and from other known references, such as Remington's Pharmaceutical Sciences, Mack Publishing Company, Easton, Pa., 18^{th} edition (1990).

For oral administration of ROCK kinase inhibitors, tablets containing the active agent are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the ROCK kinase inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration the active ROCK kinase inhibitor agent, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Additionally, it is possible to topically administer the active agent, by way of, for example, creams, lotions, jellies, gels, pastes, ointments, salves and the like, in accordance with standard pharmaceutical practice. For example, a topical formulation comprising an ROCK kinase inhibitor in about 0.1% (w/v) to about 5% (w/v) concentration can be prepared.

For veterinary purposes, the active agents can be administered separately or together to animals using any of the forms and by any of the routes described above. In a preferred embodiment, the ROCK kinase inhibitor is administered in the form of a capsule, bolus, tablet, liquid drench, by injection or as an implant. As an alternative, the ROCK kinase inhibitor can be administered with the animal feedstuff, and for this purpose a concentrated feed additive or premix may be prepared for a normal animal feed. Such formulations are prepared in a conventional manner in accordance with standard veterinary practice.

ROCK kinase inhibitors include but are not limited to low molecular weight inhibitors, peptide or RNA aptamers, antisense constructs, small inhibitory RNAs (i.e. RNA interference by dsRNA; RNAi), and ribozymes. In a preferred embodiment, the ROCK kinase inhibitor is a small organic molecule that binds specifically-to the human ROCK kinase (one or more isoforms). Specific examples of ROCK kinase inhibitors include for example Ki-23095 (Kirin Brewery Co. Ltd); WF-536 (also known as Y-32885; Mitsubishi Pharma Corp.); Y-27632 (Mitsubishi Pharma Corp.); Y-399832 (Mitsubishi Pharma Corp.); SLx-2119 (Surface Logix, Inc.); and VAS-012 (VasGene Therapeutics, Inc.); or the compounds exemplified in the following International Patent Publications: WO-2006009889; WO-2005105780; WO-2005103050; WO-2005100342; WO-2005080394; WO-2005074642; WO-2005039564; WO-2005037197; WO-2005034866; WO-2004112719; WO-2004084813; US-20040115641; WO-2004041813; WO-03080125; WO-03059913; WO-00168607; WO-00057914; U.S. Pat. Nos. 6,906,061; and 6,943172; or EPO Patent Publication No. EP 1,256,574.

In the practice of this invention many alternative experimental methods known in the art may be successfully substituted for those specifically described herein, as for example many of those described in the excellent manuals and textbooks available in the areas of technology relevant to this invention (e.g. Using Antibodies, A Laboratory Manual, edited by Harlow, E. and Lane, D., 1999, Cold Spring Harbor Laboratory Press, (e.g. ISBN 0-87969-544-7); Roe B. A. et. al. 1996, DNA Isolation and Sequencing (Essential Techniques Series), John Wiley & Sons. (e.g. ISBN 0-471-97324-0); Methods in Enzymology: Chimeric Genes and Proteins”, 2000, ed. J. Abelson, M. Simon, S. Emr, J. Thorner. Academic Press; Molecular Cloning: a Laboratory Manual, 2001, 3^{rd} Edition, by Joseph Sambrook and Peter MacCallum, (the former Maniatis Cloning manual) (e.g. ISBN 0-87969-577-3); Current Protocols in Molecular Biology, Ed. Fred M. Ausubel, et. al. John Wiley & Sons (e.g. ISBN 0-471-50338-X); Current Protocols in Protein Science, Ed. John E. Coligan, John Wiley & Sons (e.g. ISBN 0-471-11184-8); and Methods in Enzymology: Guide to protein Purification, 1990, Vol. 182, Ed. Deutscher, M. P., Acedemic Press, Inc. (e.g. ISBN 0-12-213585-7)), or as described in the many university and commercial websites devoted to describing experimental methods in molecular biology.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

Experimental Details:

Materials and Methods

Cell Lines

Human cancer cell lines were purchased from the American Type Culture Collection (ATCC). Panc-1 cells were grown in Dulbecco's modified Eagle's medium with 2 mM L-glutamine adjusted to contain 3.7 g/L sodium bicarbonate and 4.5 g/L glucose, 10% fetal bovine serum. All other cell lines were grown in media as prescribed by the ATCC, containing 10% FCS.

Antibodies and ROCK Inhibitors

The following antibodies were used for immunoblotting analysis or for antigen capture or detection in the ELISA assay: ROCK1 (Santa Cruz Biotechnology (Santa Cruz, Calif.), #sc-17794), ROCK2 (Santa Cruz Biotechnology, #sc-5561), MLC (Sigma (St. Louis, Mo.), clone MY-21, #M4401), MYPT1 (BD Transduction (San Jose, Calif.), #M38420), GAPDH (Abcam (Cambridge, Mass.), #ab9482), pMLC(S19) (Cell Signaling Technology (Danvers, Mass.), #3671), pMYPT1 (T696) (US Biologicals (Swampscott, Mass.), #M9925-09), pMYPT1(T853) (US Biologicals, #M9925-09), pCofilin(S3) (Cell Signaling Technology, #3311). The ROCK inhibitors were obtained from commercial sources: HA1077 was obtained from Sigma, H-1152 was obtained from Calbiochem (San Diego, Calif.), Y27632 was obtained from Calbiochem.

siRNA Transfection

The siRNA oligonucleotides targeting ROCK1 or ROCK2, as well as two control oligonucleotides were obtained from Invitrogen as double-stranded STEALTHTm RNAi molecules. The sequences used were as follows:

ROCK 1 sequences (sense strand):

| | |
|---|---|
| siRNA G7: | UCA GUC AGA AUU CAC AGC UUG CUA A |
| siRNA G9: | GAC AGA UGC GGG AGC UAC AAG AUC A |
| siRNA G11: | GCA UUU GGA GAA GUU CAA UUG GUA A |

ROCK 2 sequences (sense strand):

| | |
|---|---|
| siRNA H1: | GAA GCA GCU AUU AAC AGA AAG AAC A |
| siRNA H3: | CCG UUG CCA UAU UAA GUG UCA UAA A |
| siRNA H5: | GGA GGA GAU UAU AGC ACC UUG CAA A |

Low GC control siRNA: Invitrogen catalog # 12935-200

Medium GC control siRNA: Invitrogen catalog # 12935-300

Panc-1 cells were seeded into 6 well culture dishes at 300,000 cells/well and allowed to attach overnight in 2 mL growth medium prior to transfection. The cells were approximately 40% confluent at the time of transfection. siRNA oligonucleotides were diluted to 20 μM in RNAse-free water, then mixed with Lipofectamine 2000 prior to transfection (e.g. for 10 nM final [siRNA], 25 pmol oligonucleotide duplex was diluted in 250 μL serum-free culture medium, then added to 250 μL serum-free medium containing 5 μL LIPOFECTAMINE 2000® (Invitrogen, Carlsbad, Calif.). The mixture was then incubated for 20 min prior to addition to cells in 2 mL medium). Following incubation of the transfected cells for 48 h, the cultures were harvested by the addition of 150 μL of 1×SDS-PAGE sample buffer, and the lysates were analyzed by immunoblotting with appropriate antibodies.

Cell and Tumor Sample Lysis

Lysates were prepared from cultured cells, following a brief rinse in PBS, using several different buffers. Buffer A (50 mM Tris-HCl, pH 7.4, 150 mM NaCl, 10% glycerol, 1% Triton X-100, 0.5 mM EDTA, 1 mM Na4VO3, 1 mg/ml leupeptin, lmg/ml aprotinin) and buffer B (RIPA buffer: Sigma catalog #R0278, containing protease and phosphatase inhibitor cocktails Sigma catalog #P2850, P5726 P8340) were added to the cells in culture dishes, the cells were scraped from the dish and the lysates transferred to eppendorf tubes and incubated at 4° C. for 20 min. Lysates were then clarified by centrifugation at 10,000 g, and the resulting supernatants were stored at −80° C. prior to the addition of SDS-PAGE sample buffer and analysis by immunoblotting. Alternatively, lysates were prepared by the direct addition of SDS-PAGE sample buffer to the cultures, and samples were prepared for analysis by sonication for 5 min followed by heating to 100° C. for 5 min. Alternatively, lysates were prepared by the addition of PROTEOEXTRACT® solution (complete mammalian proteome extraction kit, Calbiochem, #539779) including 16.5 IU of BENZONASE® (Novagen, Inc., Madison, Wis.) followed by shaking for 30 minutes at room temperature. Samples prepared by this method were either diluted directly into SDS-PAGE sample buffer for analysis by immunoblotting, or were diluted 10-fold in buffer A for analysis in antibody capture ELISA assays. Tumor lysates were prepared using the PROTEOEXTRACT® complete mammalian proteome extraction kit (Calbiochem, #539779). Approximately 300 mg samples of tumor sample were homogenized and dispersed in 450 L resuspension buffer, then 2.4 mL extraction reagent and 150 μl reducing agent were added and the samples were mixed thoroughly. 1125U BENZONASE® was then added and the samples incubated at room temperature for 1 h followed by centrifugation at 10,000 μg for 10 min at 4° C. The resultant supernatant samples were either diluted directly into SDS-PAGE sample buffer for analysis by immunoblotting, or were diluted 10-fold in buffer A for analysis in antibody capture ELISA assays.

ELISA Assay for ROCK Inhibitors

Cells (e.g. Panc-1, etc) are seeded at an appropriate density (e.g. 15,000 cells/well) in 96-well culture plates in 90 μL of the appropriate growth medium, and allowed to incubate overnight at 37° C., 5% CO2, 95% humidity. Compounds are serially diluted to 10× final concentrations in DMSO (1%) and growth medium, then 10 μL are added to each culture well and cells are incubated with compound for 1 hour at 37° C. Cells are then washed with PBS at room temperature, and lysed by addition of 20 μL of PROTEOEXTRACT® solution (Calbiochem, #539779) including 16.5 IU of BENZONASE®. Lysis is performed with shaking for 30 minutes at room temperature. Lysates are then diluted 10-fold with Triton-containing lysis buffer (50 mM Tris-HCI, pH 7.4, 150 mM NaCl, 10% glycerol, 1% Triton X-100, 0.5 mM EDTA, 1 mM Na4VO3, 1 mg/ml leupeptin, 1 mg/ml aprotinin) and 180 μL are transferred to a 96-well capture ELISA plate. The capture plate is prepared by coating with 50 ng/well of an antibody to MYPT1 protein (e.g. BD Transduction Laboratories, mouse monoclonal antibody #M38420) in 100 μL of 0.1M sodium bicarbonate buffer (pH 9) incubated at 4° C. overnight, followed by blocking with 100 μl/well of 3% BSA in PBST for 1 h at room temperature and washing with 200 μL/well of PBST.

Following incubation of the lysates in the capture plate overnight at 4° C., the assay wells are then washed 4× with 200 μL/well of PBST prior to the addition of 100 μl of phospho-specific detection antibody (e.g. US Biologicals, pMYPT1 (T853) rabbit polyclonal antibody #M9925-08) diluted in 3% BSA in PBST, and incubation for 2 h at room temperature. The assay wells are then washed 4× with 200 μL/well of PBST prior to the addition of 100 μl of the secondary detection reagent (e.g. HRP-conjugated anti-Rabbit IgG, Jackson Laboratories #711-035-152) and incubation for 1 h at room temperature. The assay wells are then washed 5× with 200 μL/well of PBST prior to addition of appropriate detection reagent (e.g. 50 μL of SUPERSIGNAL FEMTO® luminescent HRP substrate, Pierce #37075 (Rockford, Ill.)) and quantitation using an appropriate plate reader.

Comparison of the assay signals obtained in the presence of compound with those of controls (no compound added), allows the reduction in phosphorylation of MYPT1 to be determined over a range of compound concentrations. These inhibition values are fitted to a sigmoidal dose-response inhibition curve to determine the IC₅₀ values (i.e. the concentration of compound that reduces the level of MYPT phosphorylation to 50% of the control activity).

Results and Discussion

Evaluation of ROCK1/ROCK2-Dependence of Cytoskeletal Signaling Events in Panc1 Cells Using siRNA.

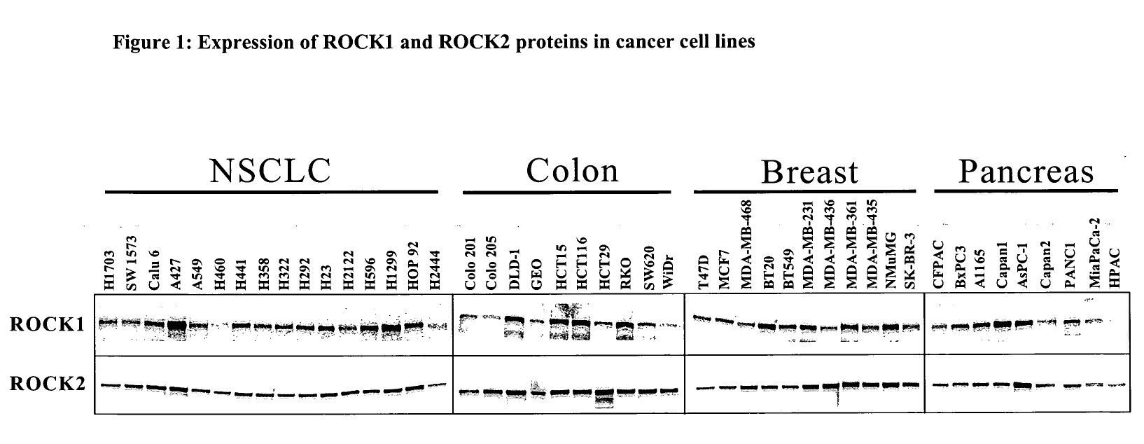

ROCK1 and ROCK2 are closely related enzymes which may have somewhat overlapping functions in various cell types (Thumkeo et al., Genes Cells 10: 825-834 (2005); Thumkeo et al., Mol. Cell. Biol. 23: 5043-5055 (2003); Shimizu et al., J. Cell Biol. 168: 941-953 (2005)). In order to evaluate the expression levels of each ROCK enzyme in cancer cell lines, a panel of cell lysates was prepared and immunoblotted for ROCK1 and ROCK2 (FIG. 1). All cell lines expressed both isoforms to some extent, although the relative expression levels were somewhat variable between cell lines.

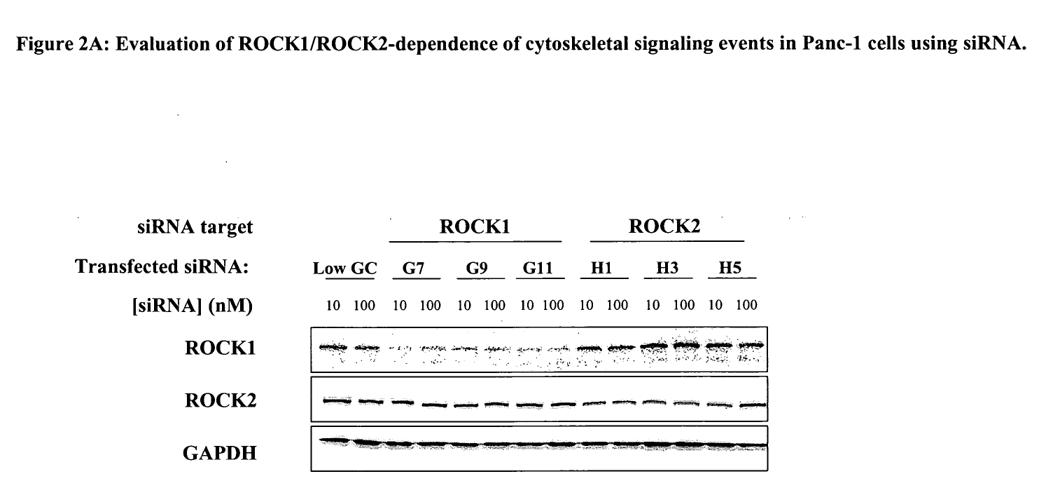

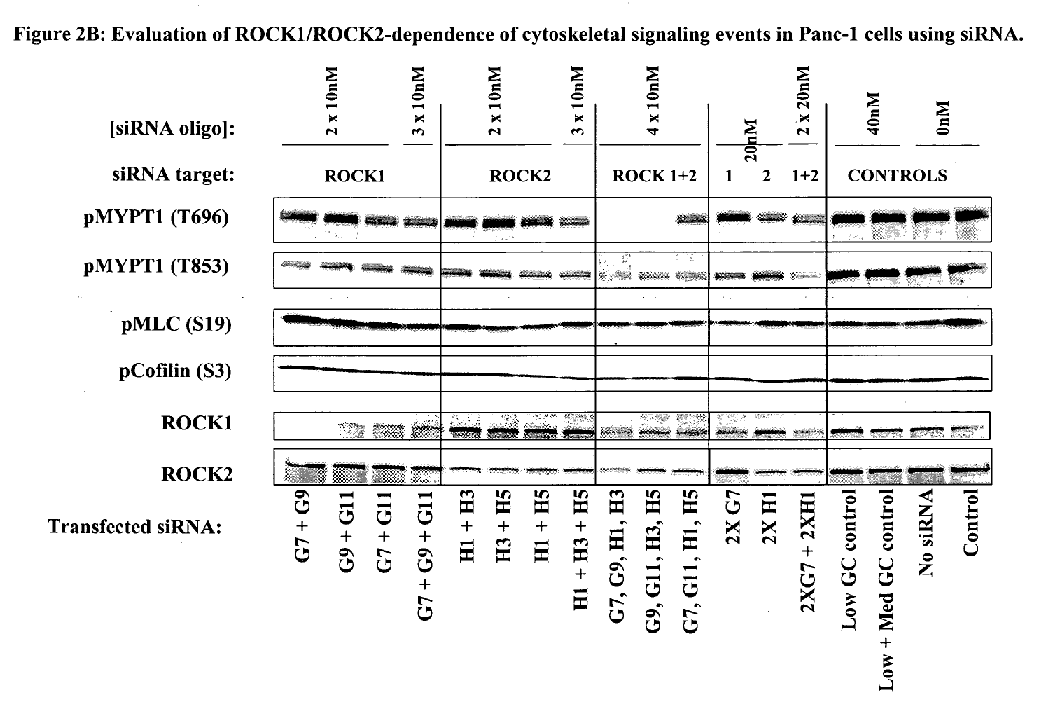

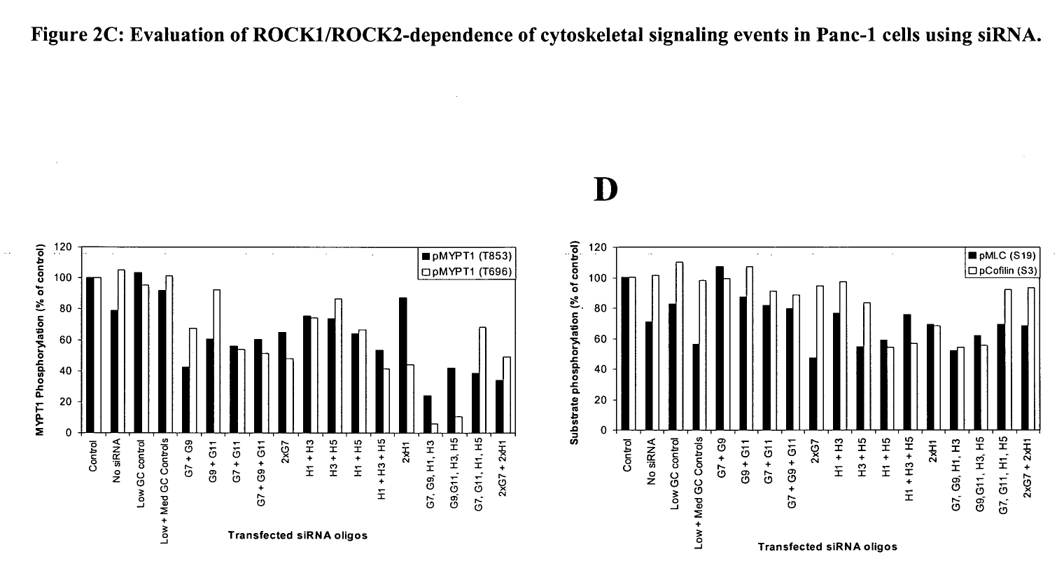

In order to evaluate the extent to which cytoskeletal signaling events are dependent on ROCK function, siRNA sequences targeting the ROCK enzymes were introduced into Panc-1 pancreatic carcinoma cells either individually or in combination. Each siRNA tested reduced significantly the expression level of ROCK1 or ROCK2 (as appropriate to the target sequence), and there was minimal difference in protein knockdown between cultures in which 10 nM or 100 nM oligonucleotide concentrations were used (FIG. 2A).

When siRNA sequences directed against ROCKi and 2 were used in combination, expression of both proteins was reduced by greater than 50% (FIG. 2B). Reduction of ROCK1 or ROCK2 protein expression alone had minimal effect on the phosphorylation state of MYPT1, MLC or cofilin, whereas targeting both ROCK1 and ROCK2 together significantly reduced MYPT1 phosphorylation at both T853 and T696 phosphorylation sites within 48 h of addition of siRNA (FIG. 2B, 2C). In contrast, there was less effect on phosphorylation of MLC or cofilin under these conditions (FIG. 2B, 2D). These data suggest that of the potential readouts evaluated MYPT1 phosphorylation represents a predominantly ROCK-dependent signaling event in Panc-1 cells, which can be effectively reduced by targeting both ROCK1 and ROCK2 enzymes but not by agents that are either specific for kinases other than ROCK, or that recognize only one of the ROCK isoforms. Monitoring the phosphorylation state of MYPT1 in Panc1 cells therefore provides a method for establishing the relative activity of the ROCK enzymes under different conditions. For example, incubation of Panc-1 cells in the-presence of novel inhibitors of ROCK may be used to establish the relative potencies of such novel compounds.

Extraction of Cytoskeletal Substrates of ROCK1/ROCK2 from Panc-1 Cell Extracts.

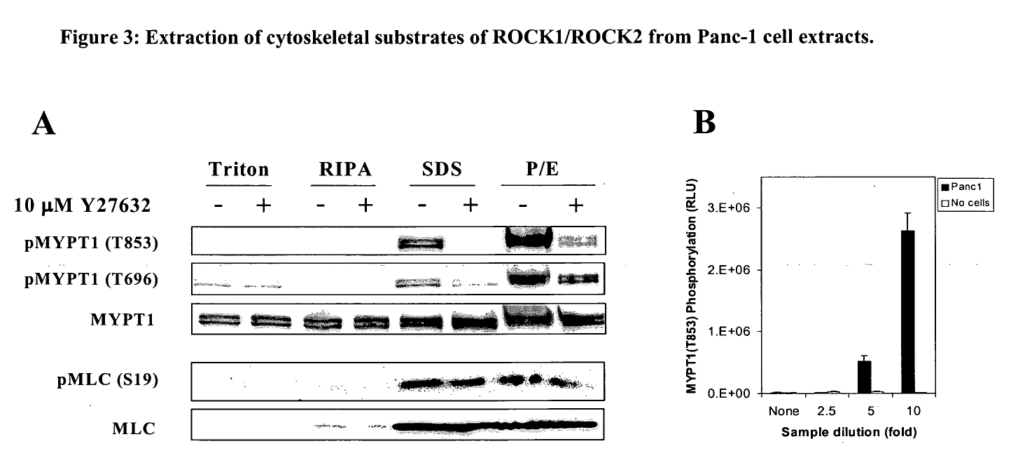

The predominant function of ROCK kinase activity is to regulate cellular morphology and migration, through controlling various aspects of cytoskeletal function. The substrates of ROCK are therefore frequently tightly associated with the insoluble fraction of cell extracts that contains cytoskeletal elements. In order to extract such proteins to evaluate the degree of protein phosphorylation it is necessary to use relatively harsh conditions (e.g. high concentrations of SDS, followed by boiling), which are incompatible with the use of such extracts in standard ELISA-based methods for determining protein phosphorylation levels. In order to develop a high-throughput mechanistic assay for ROCK inhibition in intact cells, potential cellular extraction methods were evaluated for their ability to solubilize MYPT1 and MLC (FIG. 3). These proteins were effectively extracted by boiling in SDS-PAGE sample buffer, or by the commercially available reagent PROTEOEXTRACT® (Calbiochem), whereas a minimal amount of the phosphorylated proteins was released from the insoluble fraction when standard cell lysis buffers were used (FIG. 3). Treatment of the cells with a ROCK inhibitor (Y27632) prior to lysis significantly reduced the level of MYPT1 phosphorylation observed without affecting significantly the total MYPT1 protein content of the extracts (FIG. 3).

The proteins solubilized by the PROTEOEXTRACT® reagent were not efficiently detected using standard antibody capture ELISA techniques (FIG. 3B). This is most likely because the extraction buffer contains components that prevent the interaction of the target protein (MYPT1 ) with the capture antibody. However, a significant (e.g. 10-fold) dilution of the sample in a Triton X-100 containing buffer yielded a sample that retained the extracted MYPT1 as a soluble protein, while also permitting efficient capture and detection using antibody-coated 96-well ELISA plates (FIG. 3B).

Inhibition of MYPT1 Phosphorylation by Known ROCK Inhibitor Compounds.

Titie Course of Reduction of MYPT1 Phosphorylation by Known ROCK Inhibitor Compountds.

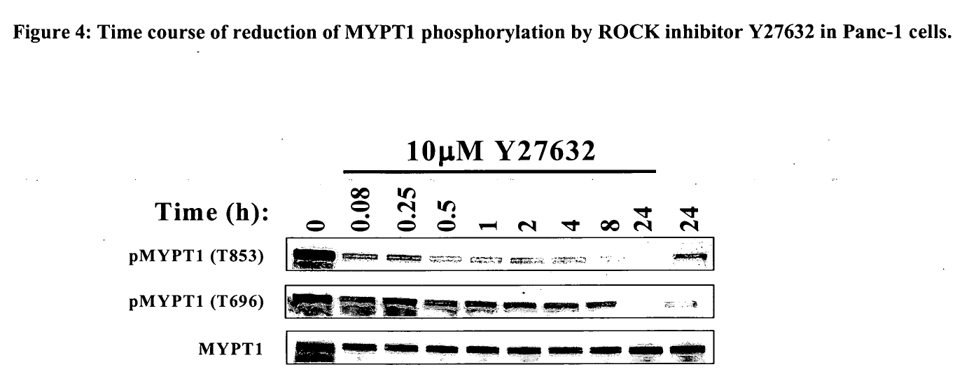

The time course of the reduction in MYPT1 phosphorylation in Panc-1 cells by the commercially available ROCK inhibitor Y27632 was evaluated by preparation of lysates from cells treated with compound for various times up to 24 h. Immunoblotting analysis revealed that the steady-state phosphorylation of MYPT1 (T853) was maximally reduced within 30-60 min of compound addition, indicating that this phosphorylation site is highly dynamic within Panc-1 cells under standard growth conditions (FIG. 4). The data further indicate that a 1 h incubation with compounds is likely to be sufficient for evaluation of the cellular potency of ROCK inhibitors. Analysis of the time course for reduction of MYPT1 T696 phosphorylation revealed that this site is somewhat less rapidly turned over under these conditions, and that therefore incubation of Panc-1 cells with ROCK inhibitors for at least 8 hours would be necessary to maximally reduce the steady-state phosphorylation of MYPT1 at this site (FIG. 4).

Potency of Inhibition of MYPT1 Phosphorylation by Known ROCK Inhibitor Compounds.

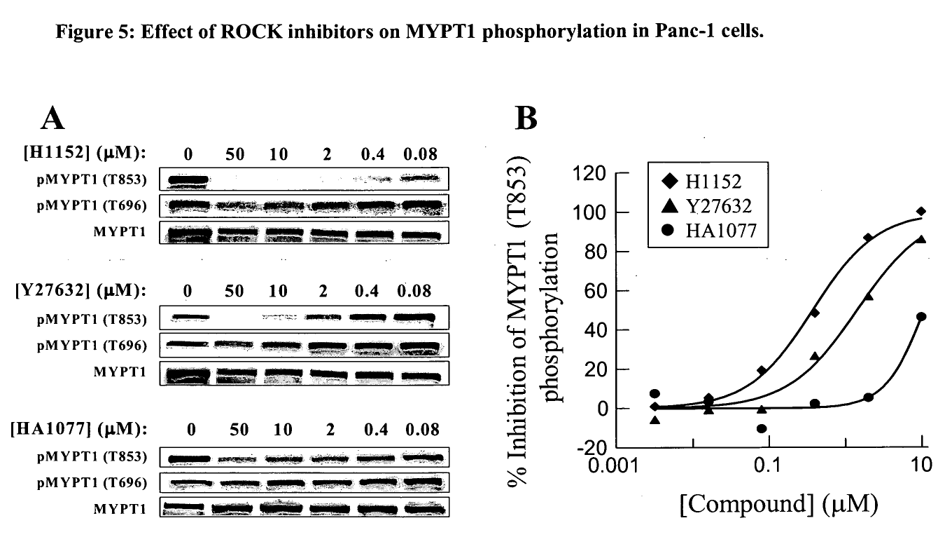

Three literature-validated ROCK inhibitors were evaluated for their ability to reduce MYPT1 phosphorylation levels during a 1 h incubation in Panc-1 cells. All three compounds effectively reduced MYPT1 phosphorylation levels at both the T853 and T696 phosphorylation sites (FIG. 5A), although the potency against the T696 phosphorylation site appeared to be lower than that observed for T853. This may be a consequence of the longer time course for reduction of this phosphorylation site (see FIG. 4). Importantly, the relative potency of the effects observed with three known ROCK inhibitors correlated well with the reported potency of these compounds in kinase inhibition assays performed with purified ROCK enzyme in vitro (Mueller, B. K. et al., Nature Rev. Drug Discovery 4: 387-396 (2005)); i.e. the rank order of potency was H1152>Y27632>HA1077, as expected. Based on these observations, a high-throughput quantitative intact cell assay for ROCK inhibitors has been developed, allowing the comparison of cellular potencies of a large number of ROCK inhibitor compounds (50-100) within 2 days.

96-Well Assay for ROCK Inhibition in Intact Cells.

The ability of compounds to inhibit ROCK activity in intact cells was determined using a 96-well antibody capture ELISA assay to detect the degree of phosphorylation of the ROCK-specific substrate MYPT1 at amino acid residue T853. In this assay format, cells are cultured in 96-well plates and incubated with compounds for 1 h prior to lysis in PROTEOEXTRACT® buffer. Lysates are then diluted 10-fold with a standard Triton X-100 containing cell lysis buffer and transferred to a second 96-well plate pre-coated with an antibody specific for MYPT1. The phosphorylation state of the captured MYPT1 is then quantitated using a phospho-specific antibody that recognizes the T853 (or T696) phosphorylation site and appropriate secondary detection reagents (e.g. an appropriate HRP-conjugated secondary antibody, followed by development with an HRP substrate). Since the level of phosphorylation of MYPT1 at T853 and T696 in Panc-1 cells appears to be largely dependent on ROCK activity (FIG. 2), the assay results can be fit to a sigmoidal dose-response curve with maximal and minimal inhibition values set at 100% and 0%, respectively in order to obtain IC₅₀ values for compound potency. Comparison of three commercially available ROCK inhibitors revealed (FIG. 5B), as expected from the immunoblotting analysis (FIG. 5A), that the rank order of compound potency in this assay correlated well with that described in the literature for inhibition of purified ROCK kinase enzyme in vitro. The signal/background ratio observed in this assay, obtained by comparing the signal strength in wells containing cell lysates to that in wells to which no lysate was added, was approximately 10-30 fold.

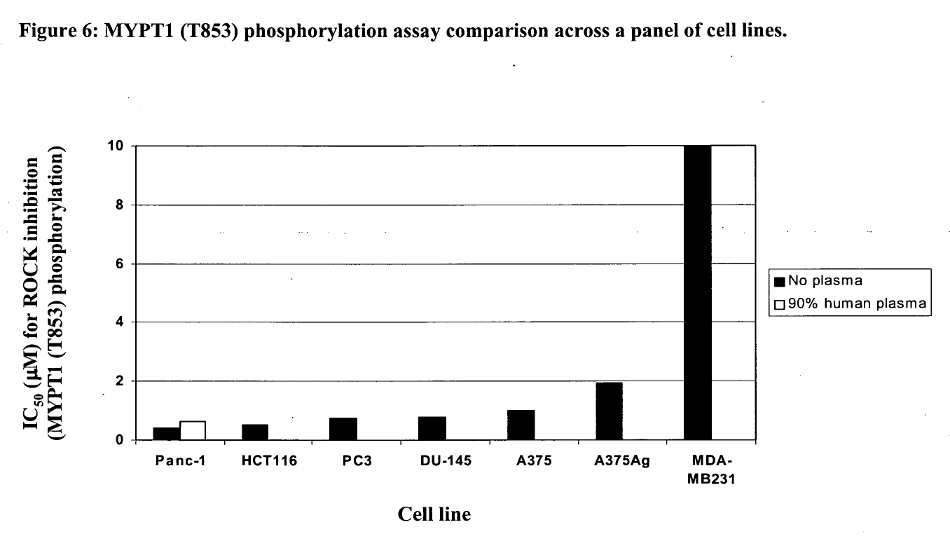

The assay for detection of MYPT1 (T853) phosphorylation was also used to compare the activity of the ROCK inhibitor H1152 across a panel of cell lines, revealing that the majority of cell lines exhibit comparable sensitivity to the ROCK inhibitor (FIG. 6). The assay for detection of MYPT1 (T853) phosphorylation in Panc-1 cells can also be performed in the presence of plasma, this allows the potential impact of plasma protein binding of novel compounds to be taken into account (FIG. 6, open bar) since this binding may interfere with the ability of novel compounds to enter cells and interact with intracellular targets such as ROCK1 and ROCK2.

Assay for ROCK Inhibition In Vivo Based on the Phosphorylation State of MYPT1.

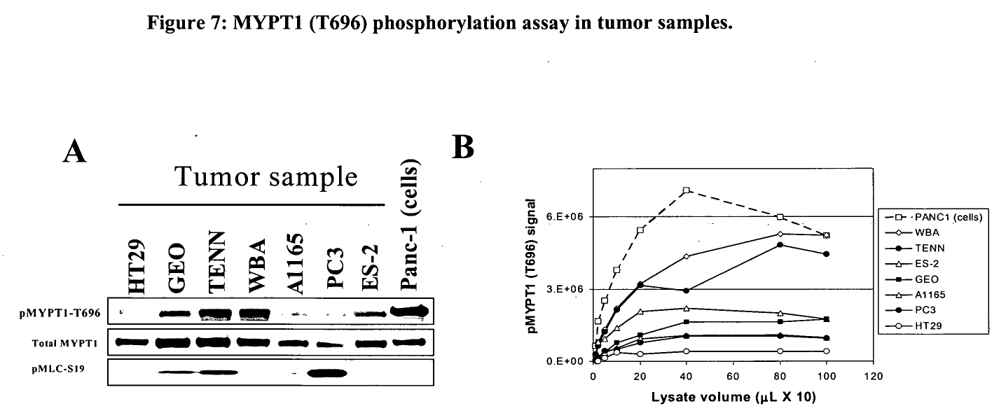

In order to evaluate the potential for quantitation of ROCK inhibition by novel compounds in vivo, lysates were prepared from a variety of tumor samples derived from human cancer cell lines grown as xenografts in immuno-compromised mice. Lysates were then evaluated for MYPT1 and MLC phosphorylation by immunoblotting. MYPT1 phosphorylation at T696 was visible in many of the tumor samples, although the signal intensity varied considerably (FIG. 7A). Certain tumor types also contained significant levels of phosphorylated MLC (S19), although this appeared in fewer samples than MYPT1 phosphorylation. The tumor lysates were also evaluated for MYPT1 T696 phosphorylation in the quantitative ELISA assay, which confirmed the relative levels of MYPT1 phosphorylation observed in the immunoblotting analysis (FIG. 7B). The data indicate that T696 phosphorylation levels may potentially be used as a quantitative biomarker to evaluate the activity of ROCK inhibitors in vivo, either in pre-clinical animal models or in clinical samples. The method is also potentially applicable to a variety of normal tissue types in which ROCK plays key roles in regulating cytoskeletal sign aling.

Abbreviations

MYPT1 (myosin binding subunit of protein phosphatase 1; also known as MBS, MLCP, protein phosphatase-1 regulatory subunit 12B, PPP1R12B); T or thr, threonine; EGF, epidermal growth factor; EGFR, epidermal growth factor receptor; EMT, epithelial-to-mesenchymal transition; NSCL, non-small cell lung; NSCLC, non-small cell lung cancer; HNSCC, head and neck squamous cell carcinoma; CRC, colorectal cancer; MBC, metastatic breast cancer; IGF-1, insulin-like growth factor-1; IC₅₀, half maximal inhibitory concentration; pY, phosphotyrosine; PI3K, phosphatidyl inositol-3 kinase; GAPDH, glyceraldehyde 3-phosphate dehydrogenase; regulatory light chain component of myosin (MLC); MAPK, mitogen-activated protein kinase; PDK-1,3-Phosphoinositide-Dependent Protein Kinase 1; mTOR, mammalian target of rapamycin; raf, protein kinase product of raf oncogene; MEK, ERK kinase, also known as mitogen-activated protein kinase kinase; ERK, Extracellular signal-regulated protein kinase, also known as mitogen-activated protein kinase; pPROTEIN, phospho-PROTEIN, “PROTEIN” can be any protein that can be phosphorylated, e.g. EGFR, ERK, S6 etc; PBS, Phosphate-buffered saline; TGI, tumor growth inhibition; WFI, Water for Injection; SDS, sodium dodecyl sulfate; SDS-PAGE, SDS-polyacrylamide gel electrophoresis; DMSO, dimethyl sulfoxide.

Incorporation by Reference

All patents, published patent applications and other references disclosed herein are hereby expressly incorporated herein by reference.

Equivalents

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A method for determining the intracellular activity of ROCK kinase comprising,
providing a sample of cells to be tested for ROCK kinase activity,
determining the level of phosphorylation of MYPT1 in the sample, and
determining the intracellular activity of ROCK kinase in the sample of cells, wherein the level of MYPT1 phosphorylation directly correlates with the level of intracellular ROCK kinase activity.

2. The method of claim 1, wherein determining the level of phosphorylation of MYPT1 in the sample is performed at the threonine 853 phosphorylation site on MYPT1.

3. The method of claim 1, wherein determining the level of phosphorylation of MYPT1 in the sample is performed at the threonine 696 phosphorylation site on MYPT1.

4. The method of claim 1, wherein determining the level of phosphorylation of MYPT1 in the sample is performed at the threonine 853 and threonine 696 phosphorylation sites on MYPT1.

5. The method of claim 1, wherein the level of phosphorylation of MYPT1 is determined using a sandwich ELISA assay in which a first antibody reagent is specific to MYPT1 protein and a second antibody reagent is specific to one or more ROCK kinase phosphorylation sites on MYPT1.

6. The method of claim 5, wherein said second antibody reagent is specific to the ROCK kinase phosphorylation site on MYPT1 at threonine 853.

7. The method of claim 5, wherein said second antibody reagent is specific to the ROCK kinase phosphorylation site on MYPT1 at threonine 696.

8. The method of claim 5, wherein said second antibody reagent is specific to the ROCK kinase phosphorylation sites on MYPT1 at threonine 853 and threonine 696.

9. The method of claim 1, wherein the level of phosphorylation of MYPT1 is determined by electrophoretic separation of the proteins in the sample of cells and immunoblot analysis using an antibody reagent specific to one or more ROCK kinase phosphorylation sites on MYPT1.

10. The method of claim 9, wherein said antibody reagent is specific to the ROCK kinase phosphorylation site on MYPT1 at threonine 853.

11. The method of claim 9, wherein said antibody reagent is specific to the ROCK kinase phosphorylation site on MYPT1 at threonine 696.

12. The method of claim 9, wherein said antibody reagent is specific to the ROCK kinase phosphorylation sites on MYPT1 at threonine 853 and threonine 696.

13. The method of claim 1, wherein the level of phosphorylation of MYPT1 is determined using an immunostaining procedure with an antibody reagent that is specific to one or more ROCK kinase phosphorylation sites on MYPT1.

14. The method of claim 13, wherein said second antibody reagent is specific to the ROCK kinase phosphorylation site on MYPT1 at threonine 853.

15. The method of claim 13, wherein said second antibody reagent is specific to the ROCK kinase phosphorylation site on MYPT1 at threonine 696.

16. The method of claim 13, wherein said second antibody reagent is specific to the ROCK kinase phosphorylation sites on MYPT1 at threonine 853 and threonine 696.

17. The method of claim 1, wherein said sample of cells is a sample of cells from cells grown in a tissue culture dish, plate or flask.

18. The method of claim 17, wherein said cells are Panc-1, HCT116, PC3, DU-145, A375, Geo, TENN, WBA, A1165, or ES-2 cells.

19. The method of claim 1, wherein said sample of cells is, or is obtained from, a tissue biopsy.

20. The method of claim 19, wherein said tissue biopsy is a tumor biopsy.

21. A method for identifying an agent that inhibits the intracellular activity of ROCK kinase comprising, providing a sample of cells having ROCK kinase activity, determining the degree of reduction of phosphorylation of MYPT 1 in the sample by contacting the sample of cells with a test agent and comparing the MYPT1 phosphorylation level with the phosphorylation level of MYPT1 in an identical control sample of cells that was not contacted with the test agent, determining the degree of inhibition of intracellular activity of ROCK kinase in the sample of cells contacted with the agent, wherein the level of MYPT 1 phosphorylation directly correlates with the level of intracellular ROCK kinase activity, and thus determining whether the test agent is an agent that inhibits the intracellular activity of ROCK kinase.

22. A method of screening a plurality of chemical compounds not known to inhibit ROCK kinase activity to identify a compound which inhibits ROCK kinase activity, which comprises contacting a sample of cells having ROCK kinase activity with the plurality of compounds not known to inhibit ROCK kinase activity, under conditions permitting inhibition by compounds known to inhibit ROCK kinase activity; determining the level of phosphorylation of MYPT1 in the sample; determining the intracellular activity of ROCK kinase in the sample of cells, wherein the level of MYPT1 phosphorylation directly correlates with the level of intracellular ROCK kinase activity; comparing the intracellular activity of ROCK kinase in the sample of cells with that in an identical control sample of cells that had not been treated with the plurality of compounds; and where inhibition of ROCK kinase activity by the plurality of compounds is observed, separately determine the inhibition of ROCK kinase activity of each compound included in the plurality of compounds, so as to thereby identify any individual compound included therein which inhibits ROCK kinase.

23. A method of preparing a composition comprising a chemical compound which inhibits ROCK kinase activity, which comprises identifying an chemical that inhibits the intracellular activity of ROCK kinase comprising, providing a sample of cells having ROCK kinase activity, determining the degree of reduction of phosphorylation of MYPT1 in the sample by contacting the sample of cells with a test chemical, determining the degree of inhibition of intracellular activity of ROCK kinase in the sample of cells, wherein the level of MYPT1 phosphorylation directly correlates with the level of intracellular ROCK kinase activity, identifying the test chemical as a chemical that inhibits the intracellular activity of ROCK kinase, and admixing the test chemical so identified, or a functional analog or homolog of said test chemical, with a carrier, thereby preparing said composition.
